(19) United States
(12) Patent Application Publication
(10) Pub. No.: US 2011/0238150 A1
(43) Pub. Date: Sep. 29, 2011

(54) Bioerodible Medical Implants

(75) Inventors: Charles DENG, Chanhassen, MN (US); Liliana ATANASOSKA, Edina, MN (US); Jonathan S. STINSON, Plymouth, MN (US); Anthony O'CONNOR, St. Louis Park, MN (US); Pankaj GUPTA, Minnetonka, MN (US)

(73) Assignee: Boston Scientific SciMed, Inc., Maple Grove, MN (US)

(21) Appl. No.: 13/053,519

(22) Filed: Mar. 22, 2011

Related U.S. Application Data
(60) Provisional application No. 61/316,722, filed on Mar. 23, 2010.

(51) Int. Cl. A61F 2/82; C22C 1/10; C22C 33/00; C22C 38/18

(52) U.S. Cl. 623/1.15; 75/392

(57) ABSTRACT

An implantable medical device includes a bioerodible portion adapted to erode when exposed to a physiological environment. The bioerodible portion includes an alloy comprising at least 10 weight percent chromium and has an outer surface having a ratio of chromium oxide to chromium metal of less than 5. The bioerodible implantable medical device can be created by implanting metallic ions into an alloy including at least 10 weight percent chromium to define an outer surface of a medical implant, or precursor thereof.

### CROSS-REFERENCE TO RELATED APPLICATIONS

This application claims priority under 35 U.S.C. §119(e)(1), to U.S. Provisional Application Ser. No. 61/316,722, filed on Mar. 23, 2010.

### TECHNICAL FIELD

This invention relates to bioerodible medical implants, such as stents.

### BACKGROUND

A medical implant can replace, support, or act as a missing biological structure. Examples of medical implants include: orthopedic implants; bioscaffolding; endoprostheses such as stents, covered stents, and stent-grafts; bone screws; and aneurism coils. Some medical implants are designed to erode under physiological conditions.

Endoprostheses are typically tubular implants that can be implanted in various passageways in a body, such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

### SUMMARY

An implantable medical device is described that includes a bioerodible portion adapted to erode when exposed to a physiological environment. The bioerodible portion includes an alloy comprising at least 10 weight percent chromium and has an outer surface having a ratio of chromium oxide to chromium metal of less than 5.

The alloy, in some embodiments, can include iron. For example, the alloy can be a stainless steel alloy. The outer surface of the alloy can include a chromium-to-iron ratio of less than 0.9. The chromium to iron ratio can, in some embodiments, be less than 0.7. For example, the outer surface of the alloy can be treated to have a chromium-to-iron ratio of between 0.1 and 0.5. In other embodiments, the alloy can be substantially free of iron. For example, in some embodiments, the alloy is a cobalt-chromium alloy (e.g., L605 or MP35N).

In some embodiments, the alloy can be a low nickel alloy. For example, the alloy can include less than 0.3 weight percent nickel. In other embodiments, the alloy can be a low nickel stainless steel.

The alloy, in some embodiments, can include at least 15 weight percent chromium. For example, the alloy, in some embodiments, is a stainless steel alloy including between 16 and 25 weight percent chromium. In other embodiments, the alloy can include a cobalt-chromium alloy including between 15 and 30 weight percent chromium.

The implantable medical device can include a coating over the bioerodible metal portion. The coating can include fissures or other openings to allow for electrolyte to contact the bioerodible metal portion to create a galvanic couple between the bioerodible metal portion and the coating.

The implantable medical device can be an endoprosthesis. In some embodiments, the medical implant is a stent

In another aspect, a method of forming a bioerodible implantable medical device is described. The method includes implanting metallic ions into an alloy, which includes at least 10 weight percent chromium, to define an outer surface of a medical implant, or precursor thereof, to reduce a ratio of chromium oxide to chromium metal at the outer surface to less than 5. In some embodiments, the step of implanting the metallic ions reduces a chromium-to-iron atomic ratio in the outer surface of the alloy to 0.4 or less. The metallic ions can be selected from the group consisting of iron and iridium ions. The outer surface can include between 0.1 and 10 atomic percent of the implanted metallic ions after implanting the metallic ions into the alloy. The medical implant can be, for example, a stent.

The details of one or more embodiments are set forth in the accompanying drawings and the description below. Other features, objects, and advantages will be apparent from the description and drawings, and from the claims.

### DESCRIPTION OF DRAWINGS

FIG. 1 is a perspective view of an example of an expanded stent.

FIGS. 2A and 2B depict an example of a treated 316L stainless steel stent.

FIGS. 3A-3C depict an example of a treated 316L stainless steel stent having an outer layer of iridium oxide.

Like reference symbols in the various drawings indicate like elements.

### DETAILED DESCRIPTION

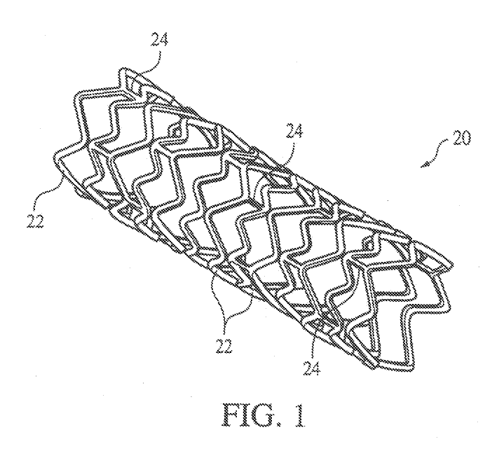

The medical implant includes a bioerodible alloy. A stent 20, shown in FIG. 1, is discussed below as an example of one medical implant. Other examples of medical implants can include: orthopedic implants; bioscaffolding; bone screws; aneurism coils, heart valves; implant filters; and other endoprostheses such as covered stents and stent-grafts.

As shown in FIG. 1, stent 20 includes a pattern of interconnected struts forming a structure that contacts a body lumen wall to maintain the patency of the body lumen. For example, stent 20 can have the form of a tubular member defined by a plurality of bands 22 and a plurality of connectors 24 that extend between and connect adjacent bands. During use, bands 22 can be expanded from an initial, small diameter to a larger diameter to contact stent 20 against a wall of a vessel, thereby maintaining the patency of the vessel. Connectors 24 can provide stent 20 with flexibility and conformability that allow the stent to adapt to the contours of the vessel.

Many alloys including at least 10 weight percent chromium have particular advantages for use in medical implants (e.g., suitable mechanical properties), but medical implants using these alloys have naturally passivateized surfaces due to the reaction of the chromium on the surface of the alloy and the environment resulting in the formation of a thin layer of corrosion product that passivates the surface and limits further corrosion. These naturally passivated alloys are accordingly biostable.

Stent 20, however, includes at least one bioerodible portion adapted to erode when exposed to a physiological environment that includes an alloy having at least 10 weight percent chromium; e.g., 300-series austenitic stainless steels, L605, or MP35N. The alloy, in some embodiments, has a chromium content of at least 11.5 weight percent. In some embodiments, the alloy has a chromium content of at least 15 weight percent. For example, the chromium content can be between 16 and 30 weight percent (e.g., between 18 and 25 weight percent). To ensure that the alloy is bioerodible, as opposed to biostable, an outer surface of the alloy has a chromium oxide to chromium metal ratio of less than 5. This ratio is expressed in terms of the ratio of number of chromium atoms that are oxidized to the number of chromium atoms that are metallic. For the purposes of this application, the outer surface of the alloy is defined as an outer 10 micrometer thickness of the alloy. The thickness of the layer is measured by XPS or auger electron depth profiling spectroscopy. The depth is the perpendicular distance from the surface to the interior. An outer surface having a chromium oxide to chromium metal ratio of less than 5 can ensure that the alloy erodes in a physiological environment. The erosion occurs first in the outer layer and produces crevices that perpetuate crevice corrosion into the interior. In some embodiments, the chromium oxide to chromium metal ratio is less than 4. In still other embodiments, the chromium oxide to chromium metal ratio is less than 3.5. For example, the chromium oxide to chromium metal can be about 2.

An outer surface having a chromium oxide to chromium metal ratio of less than 5 experiences less corrosion resistance than a naturally passivated chromium containing alloy. Chromium containing alloys become naturally passivated due to the reaction of the chromium on the surface of the alloy and the environment resulting in the formation of a thin layer of chromium oxide that passivates the surface and limits further corrosion. By reducing the amount of chromium oxide present in the outer surface such that the ratio is less than 5 can make the alloy bioerodible. Once the outer surface erodes, the remainder of the alloy can erode due to the presence of chloride within the physiological environment that causes intergranular corrosion, galvanic corrosion, pitting corrosion, and/or crevice corrosion. One advantage of this is that bioerodible implants can be made from existing stainless alloy designs by treating the surface to cause chromium-depletion, rather than having to redesign the implant utilizing a different material such as a bioabsorbable polymer or metal.

Table I, below, depicts a comparison of the compositions of a bare metal naturally passivated stent with a plasma treated stent having the same starting composition. OD indicates that the sample was taken along the outer diameter, while CF indicates that the sample was taken along a cut face of the stent (e.g., the side of the struts). Table II depicts the elemental compositions of these samples in atomic percentages. Both stents include a 316L stainless steel alloy; the plasma cleaned stents include iridium in the outer layer due to the plasma cleaning process.

TABLE I

| | | | | | Cr | Fe | Cr | Cr | |
|---|---|---|---|---|---|---|---|---|---|
| | | Cr | | Fe | Ox- | Ox- | Metal/ | Oxide/ | Ox- |
| | Cr | ox- | Fe | ox- | ide/ | ide/ | Fe | Fe | ide/ |
| Sample | metal | ide | metal | ide | Cr | Fe | Metal | Oxide | Metal |
| Bare, | 11 | 89 | 68 | 33 | 7.98 | 0.48 | 0.16 | 2.73 | 1.54 |
| OD | | | | | | | | | |
| Bare, | 13 | 87 | 64 | 36 | 6.79 | 0.56 | 0.20 | 2.44 | 1.59 |
| CF | | | | | | | | | |
| Plasma | 22 | 78 | 31 | 69 | 3.46 | 2.19 | 0.72 | 1.13 | 2.72 |
| Clean, | | | | | | | | | |
| OD | | | | | | | | | |
| Plasma | 33 | 67 | 39 | 61 | 2.01 | 1.58 | 0.86 | 1.09 | 1.78 |
| Clean, | | | | | | | | | |
| CF | | | | | | | | | |

TABLE II

| Sample/Area | B | C | O | Si | Cr | Fe | Ni | Mo | Ir | Cr/Fe | O/Metal* | O/Cr** | O/Fe** |
|---|---|---|---|---|---|---|---|---|---|---|---|---|---|
| Bare, OD | 2.4 | 18.3 | 50.2 | 1.1 | 13.3 | 9.5 | 4.2 | 1 | 0 | 1.40 | 1.79 | 3.77 | 5.28 |
| Bare, CF | 1.6 | 18.7 | 48.9 | 0.9 | 13.6 | 11.3 | 4.2 | 0.9 | 0 | 1.20 | 1.63 | 3.60 | 4.33 |
| Plasma Clean, | 1.7 | 15 | 50.2 | — | 4.4 | 18.8 | 4.8 | 0.6 | 4.6 | 0.23 | 1.51 | 11.41 | 2.67 |
| OD | | | | | | | | | | | | | |
| Plasma Clean, | 1.7 | 19.5 | 44.3 | — | 4.8 | 20.1 | 4.8 | 0.7 | 4.3 | 0.24 | 1.28 | 9.23 | 2.20 |
| CF | | | | | | | | | | | | | |

As shown in Table I, the naturally passivated bare metal stent has a chromium oxide to chromium metal content of about 8.0 along the outer diameter and a chromium oxide to chromium metal ratio of about 6.8 along a cut face. A plasma cleaned stent, however, has a reduced chromium oxide to chromium metal ratio of about 3.5 along the outer diameter and a chromium oxide to chromium metal ratio of about 2.0 along a cut face of the stent. The plasma cleaned stent also has an increased ratio of iron oxide to iron metal and a decreased ratio of chromium to iron.

An outer surface of the alloy can also be chromium-depleted relative to a passivated surface. Because of the presence of substantial oxygen, and potentially other passivating elements (e.g., nitrogen), at the outer passivated surface, it is useful to express the chromium content as a ratio of chromium to other constituents of the alloy. For example, as shown above, a bare metal naturally passivated surface of a 316L stainless steel stent can have an atomic percentage of about 13-14 percent chromium, but the surface also includes significant amounts of oxygen. Ignoring the oxygen however, the atomic percentage of chromium relative to the other non-oxygen constituents is about 26-28 atomic percent. This compares to the 17 to 20 atomic percent of chromium in 316L stainless steel; accordingly a naturally passivated stainless steel has an increased percentage of chromium relative to the other constituents of the alloy.

For steels, it is useful to use the atomic ratio of chromium to iron. A passivated 316L stainless steel surface commonly has a chromium-to-iron atomic ratio of greater than 1.0, as shown in Table II. By reducing the chromium to iron atomic ratio to less than 0.9, the erosion resistance of the surface can be reduced. For example, Table II shows a plasma treated stent having a surface layer having an atomic ratio of chromium to iron of about 0.25. By reducing the chromium content of the outer surface, the erosion resistance of the surface is reduced. In some embodiments, the outer surface can have a chromium percent to iron ratio of less than 0.7. For example, the outer surface can have a chromium percent to iron ratio of between 0.1 and 0.5.

The alloy can include iron. The alloy can have the composition of a stainless steel, but have a surface having a chromium to iron atomic ratio of 0.4 or less (e.g., between 0.05 and 0.3). The atomic ratio of chromium to iron can be determined by Auger electrospectroscopy and x-ray photoelectron spectroscopy analysis. Stainless steel implants generally have a passivated surface having a chromium-to-iron atomic ratio of at least 0.9. As will be discussed below, the chromium to iron atomic ratio of a stainless steel surface can be altered by physical vapor deposition (PVD) processes. Examples of stainless steel compositions that can be used as a bioerodible metal include 316L stainless steel, 316 stainless steel, 304L stainless steel, 304 stainless steel, and 302 stainless steel. The typical compositions of these stainless steel alloy compositions are shown below in Table III.

TABLE III

| Amounts in | | | | | |
|---|---|---|---|---|---|
| Weight % | 316L SS | 316 SS | 304L SS | 304 SS | 302 SS |
| Chromium | 16-18 | 16-18 | 18-20 | 18-20 | 17-19 |
| Nickel | 10-14 | 10-14 | 8-12 | 8-10.5 | 8-10 |
| Molybdenum | 2-3 | 2-3 | — | — | — |
| Manganese | ≦2 | ≦2 | ≦2 | ≦2 | ≦2 |
| Silicon | ≦0.75 | ≦0.75 | ≦0.75 | ≦0.75 | ≦1 |
| Phosphorous | ≦0.045 | ≦0.045 | ≦0.045 | ≦0.045 | ≦0.045 |
| Sulfur | ≦0.03 | ≦0.03 | ≦0.03 | ≦0.03 | ≦0.03 |
| Carbon | ≦0.03 | ≦0.08 | ≦0.03 | ≦0.08 | ≦0.15 |
| Iron | Balance | Balance | Balance | Balance | Balance |

The alloy can have less than 0.3 weight percent nickel. For example, the nickel-free stainless steel compositions described in U.S. Pat. No. 6,508,832, which is hereby incorporated by reference, can also be used to as the alloy having a chromium depleted surface. In other embodiments, the alloy includes more than 5 weight percent nickel (e.g., between 10 weight percent and 35 weigh percent).

The alloy can include cobalt. In some embodiments, the alloy is a cobalt-chromium alloy or a cobalt-nickel-chromium alloy. For example, the alloy can have the composition of L605, which includes 19-21 weight percent chromium, 14-16 weight percent tungsten, 9-11 weight percent nickel, less than 3 weight percent iron, 1-2 percent manganese, 0.05-0.015 weight percent carbon, less than 1 weight percent silicon, less than 0.04 weight percent phosphorous, less than 0.03 weight percent sulfur, and a balance of cobalt. The alloy can also the composition of MP35N®, which has about 20 weight percent chromium, about 35 weight percent nickel, about 10 weight percent molybdenum, and about 35 weight percent cobalt.

The medical implant can also include a metal or metal oxide that creates a galvanic couple with the alloy to facilitate the corrosion of the alloy. In the presence of ion-containing fluids such as plasma and blood, the alloy and a more noble metal or metal oxide can form an electrochemical cell in which the alloy acts as an anode, the more noble metal or metal oxide acts as a cathode, and the fluid acts as an ion-conducting electrolyte. The metal or metal oxide can be in discrete locations and/or overly the alloy surface, with fissures or entrances that allow the electrolyte to communicate with the alloy surface. Accordingly, when exposed to a physiological environment of a body passageway, the chromium-depleted alloy is oxidized and releases electrons that travel to the more noble metal or metal oxide to allow for a reduction reaction. The oxidation reaction can accelerate the erosion of the alloy. Suitable materials for forming a galvanic couple with the alloy include, for example, noble metals such as platinum, iridium, and ruthenium, as well as oxides of these metals (e.g., iridium oxide) and refractory metals such as titanium, hafnium, zirconium, and niobium, and oxides thereof. These materials can be deposited onto the medical implant using physical vapor deposition (PVD), electroplating, and/or chemical vapor deposition (CVD) processes.

The alloy of the bioerodible portion can be made bioerodible by reducing the chromium oxide to chromium metal ratio of the outer surface of the bioerodible portion to less than 5 by incorporating a material within an alloy. These processes can also reduce the chromium content of the surface. For example the material can be incorporated into the alloy using a physical vapor deposition (PVD) process. In other embodiments, ion beam surface treatment can be used to reduce the chromium content of the outer surface. In some embodiments, between 0.1 and 10 atomic percent of the material is deposited into the outer 10 micrometers of the alloy. The material can be selected from the group consisting of iron, iridium, sulfur, platinum, ruthenium, titanium, hafnium, zirconium, niobium, and oxides thereof. In some embodiments, depositing the material into a stainless steel alloy can reduce the chromium to iron atomic ratio to 0.3 or less.

The alloy can have an erosion rate of at least 70 μg/day when exposed a phosphate buffered 0.14 M saline solution maintained at body temperature (e.g., about 37 degrees Celsius).

The stent can include a therapeutic agent. In some embodiments, a therapeutic agent can be deposited over the outer surface of the alloy, be incorporated into a drug-eluting coating overlying the alloy, or be included in or on another portion of the medical implant. The term “therapeutic agent” includes one or more “therapeutic agents” or “drugs.” The terms “therapeutic agents” and “drugs” are used interchangeably and include pharmaceutically active compounds, nucleic acids with and without carrier vectors such as lipids, compacting agents (such as histones), viruses (such as adenovirus, adeno-associated virus, retrovirus, lentivirus and a-virus), polymers, antibiotics, hyaluronic acid, gene therapies, proteins, cells, stem cells and the like, or combinations thereof, with or without targeting sequences. The delivery mediated is formulated as needed to maintain cell function and viability. An example of a therapeutic agent includes Paclitaxel.

Stent 20 can be of any desired shape and size (e.g., superficial femoral artery stents, coronary stents, aortic stents, peripheral vascular stents, gastrointestinal stents, urology stents, and neurology stents). Depending on the application, the stent can have a diameter of between, for example, 1 mm to 46 mm. In certain embodiments, a coronary stent can have an expanded diameter of from 2 mm to 6 mm. In some embodiments, a peripheral stent can have an expanded diameter of from 5 mm to 24 mm. In certain embodiments, a gastrointestinal and/or urology stent can have an expanded diameter of from 6 mm to about 30 mm. In some embodiments, a neurology stent can have an expanded diameter of from about 1 mm to about 12 mm. An Abdominal Aortic Aneurysm (AAA) stent and a Thoracic Aortic Aneurysm (TAA) stent can have a diameter from about 20 mm to about 46 mm.

In use, a stent can be used, e.g., delivered and expanded, using a catheter delivery system. Catheter systems are described in, for example, Wang U.S. Pat. No. 5,195,969, Hamlin U.S. Pat. No. 5,270,086, and Raeder-Devens, U.S. Pat. No. 6,726,712. Stents and stent delivery are also exemplified by the Sentinol® system, available from Boston Scientific Scimed, Maple Grove, Minn. In some embodiments, the expansion of the stent during delivery can create fissures and/or cracks in the outer surface of the alloy which can facilitate a galvanic corrosion of the outer surface of the alloy.

In some embodiments, stents can also be a part of a covered stent or a stent-graft. In other embodiments, a stent can include and/or be attached to a biocompatible, non-porous or semi-porous polymer matrix made of polytetrafluoroethylene (PTFE), expanded PTFE, polyethylene, urethane, or polypropylene.

In some embodiments, medical implants other than stents include: orthopedic implants; bioscaffolding; bone screws; aneurism coils; heart valves; implant filters; and other endoprostheses such as covered stents and stent-grafts. These medical implants can be formed of a bioerodible metal and include a coating including a matrix of a fatty acid salt having metallic nano-particles within the matrix.

### EXAMPLES

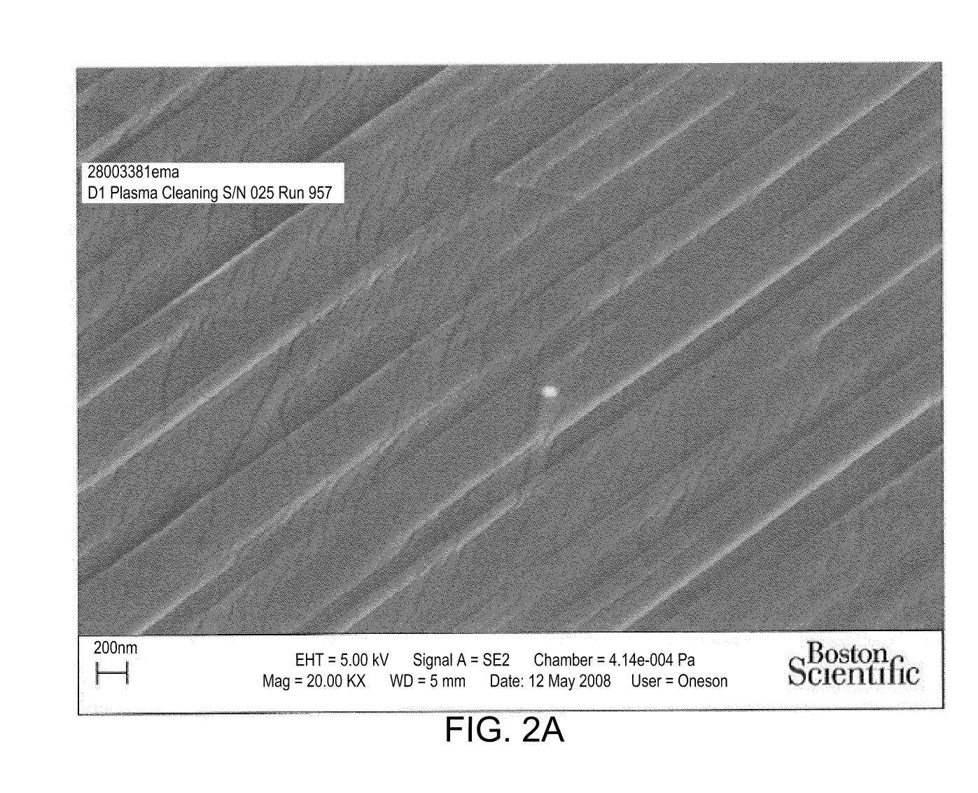

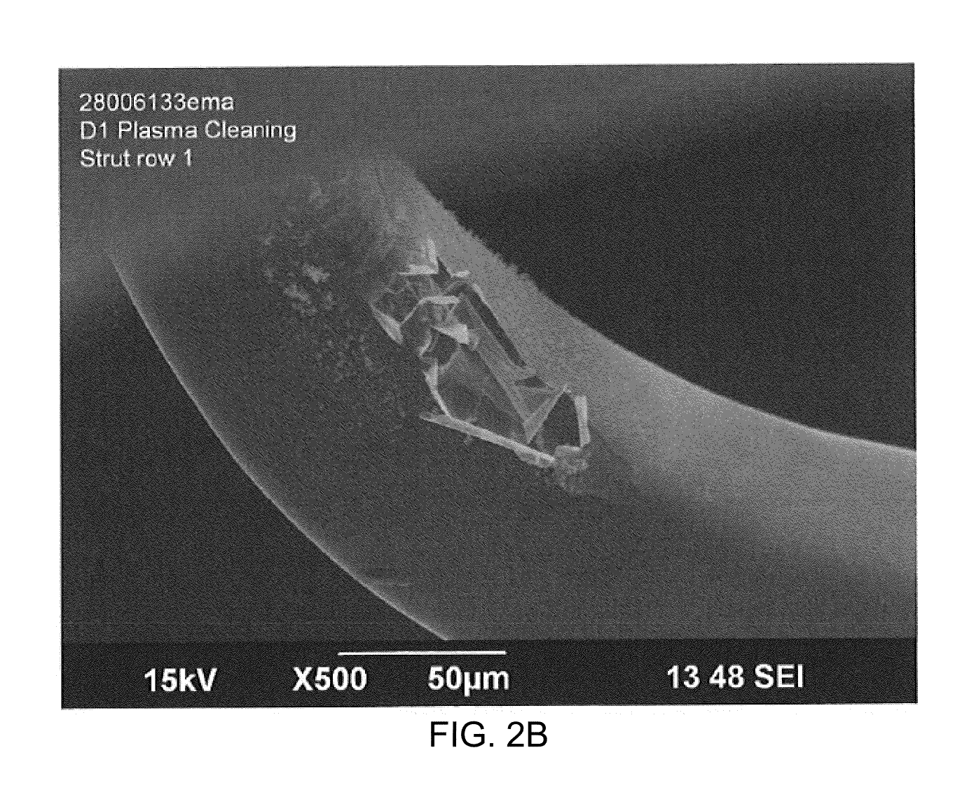

A 316L stainless steel stent body was treated in a “plasma cleaning” method where iron ions were vaporized off of the PVD chamber components and implanted into the surface of the stent body to reduce the chromium content of the outer surface. As shown in FIG. 2A, the outer surface of the stent body showed fissures after the expansion of the treated stent. As shown in FIG. 2B, the stent showed evidence of corrosion after a potentiodynamic polarization test according to the ASTM F2129 test method.

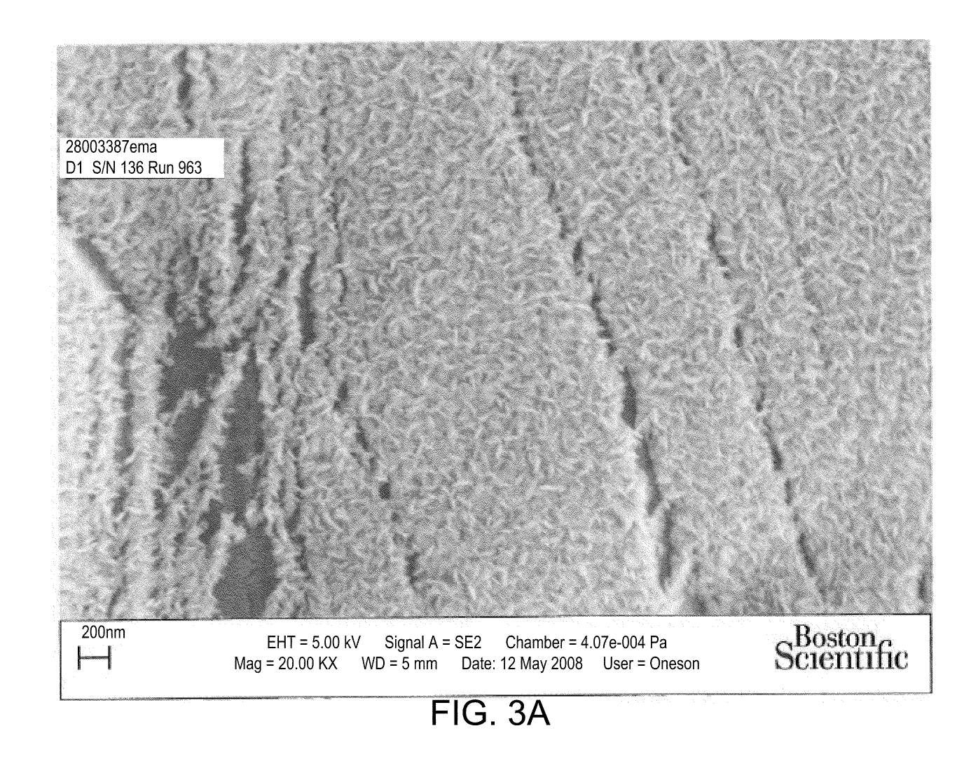

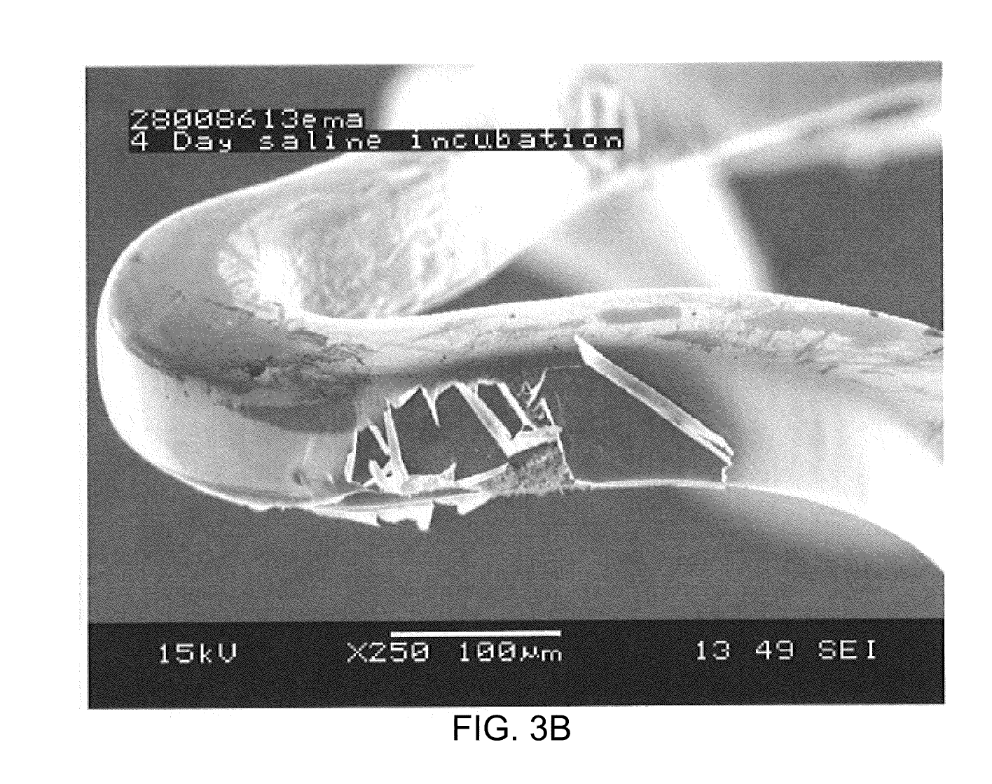

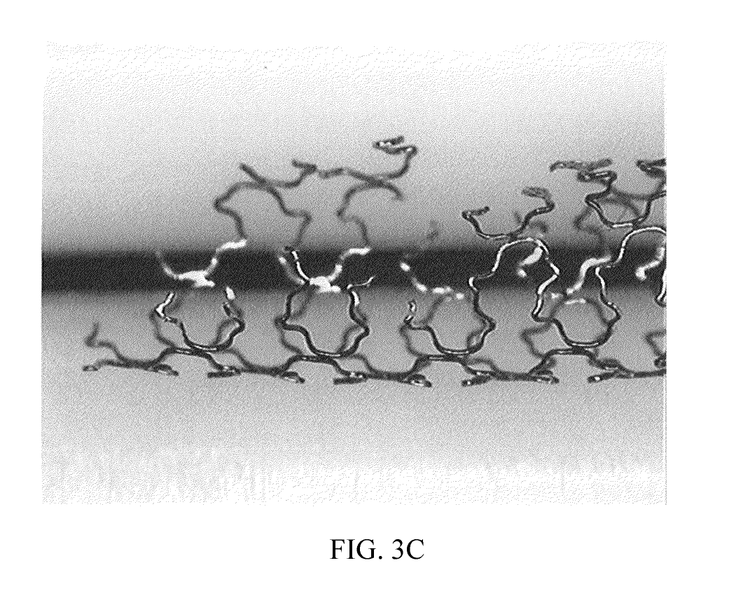

A second 316L stainless steel stent body was again treated in the above discussed “plasma cleaning” method and that coated with iridium oxide in a PVD deposition process. As shown in FIG. 3A, cracks appear in the iridium oxide coating upon stent expansion. As shown in FIG. 3B, the stent showed evidence of corrosion after 4 days of a potentiodynamic polarization test in a phosphate buffered 0.14M saline solution. FIG. 3C depicts a plasma treated 316L stainless steel stent having iridium deposited after a potentiodynamic polarization test according to the ASTM F2129 test. Other embodiments are within the scope of the claims.

What is claimed is:

1. An implantable medical device comprising a bioerodible metal portion adapted to erode when exposed to a physiological environment, the bioerodible metal portion including an alloy comprising at least 10 weight percent chromium, an outer surface of the bioerodible metal portion comprising a ratio of chromium oxide to chromium metal of less than 5.

2. The implantable medical device of claim 1, wherein the alloy comprises iron.

3. The implantable medical device of claim 2, wherein the outer surface comprises a chromium-to-iron atomic ratio of less than 0.9.

4. The implantable medical device of claim 2, wherein the alloy is a stainless steel alloy.

5. The implantable medical device of claim 1, wherein the alloy is a cobalt-chromium alloy.

6. The implantable medical device of claim 1, wherein the alloy is a cobalt-chromium alloy.

7. The implantable medical device of claim 1, wherein the alloy comprises less than 0.3 weight percent nickel.

8. The implantable medical device of claim 1, wherein the alloy comprises at least 15 weight percent chromium.

9. The implantable medical device of claim 1, further comprising a coating over the bioerodible metal portion.

10. The implantable medical device of claim 1, wherein the implantable medical device is an endoprosthesis.

11. The implantable medical device of claim 1, wherein the implantable medical device is a stent.

12. A method of forming a bioerodible implantable medical device, comprising:
implanting metallic ions into an alloy defining an outer surface of a medical implant, or precursor thereof, to reduce a ratio of chromium oxide to chromium metal in the outer surface to less than 5, the alloy comprising at least 10 weight percent chromium.

13. The method of claim 12, wherein the alloy is a stainless steel alloy and implanting the metallic ions into the alloy reduces a chromium-to-iron atomic ratio in the outer surface of the alloy to 0.4 or less.

14. The method of claim 12, wherein the metallic ions are selected from the group consisting of iron and iridium ions.

15. The method of claim 12, wherein the outer surface comprises between 0.1 and 10 atomic percent of the implanted metallic ions after implanting the metallic ions into the alloy.

16. The method of claim 12, wherein the medical implant is a stent.
